(19) United States
(12) Patent Application Publication
(10) Pub. No.: US 2013/0197279 A1
(43) Pub. Date: Aug. 1, 2013

(54) Water and Contaminants Removal from Butanol Fermentation Solutions and/or Broths Using a Brine Solution

(71) Applicant: Michael E. Wright, Ridgecrest, CA (US)

(72) Inventor: Michael E. Wright, Ridgecrest, CA (US)

(21) Appl. No.: 13/433,737

(22) Filed: Mar. 29, 2012

Related U.S. Application Data
(63) Continuation-in-part of application No. 12/511,796, filed on Jul. 29, 2009, now Pat. No. 8,395,007.
Application No. 12/511,796 is a continuation-in-part of application No. 12/550,973, filed on Aug. 31, 2009, now Pat. No. 8,227,651.
(60) Provisional application No. 61/562,231, filed on Nov. 21, 2011.

(51) Int. Cl. C07C 29/80; C07C 31/12; C12M 1/00

(52) U.S. Cl. 568/913; 435/289.1

(57) ABSTRACT

A process for removal of water and other selected containments from butanol fermentation broths which is achieved by continuous extraction with a concentrated salt solution with a regulated pH value. This process greatly reduces distillation energy and losses for the final purification of butanol (normal or iso-). Furthermore, this process allows for generation of sufficiently pure biobutanol (>97%) for use in a subsequent chemical process.

### CROSS-REFERENCE TO RELATED APPLICATIONS

This is a continuation-in-part, claiming the benefit of, parent application Ser. No. 12/511,796 filed on Jul. 29, 2009 and Ser. No. 12/550,973 filed on Aug. 31, 2009, whereby the entire disclosure of which is incorporated hereby reference.

### STATEMENT REGARDING FEDERALLY SPONSORED RESEARCH OR DEVELOPMENT

The invention described herein may be manufactured and used by or for the government of the United States of America for governmental purposes without the payment of any royalties thereon or therefor.

### FIELD OF THE INVENTION

The invention generally relates to a fuel and process for dewatering and purifying butanol prepared through a fermentation process.

### BRIEF DESCRIPTION OF THE DRAWING

FIG. 1. A general overview of process going from sugars (fermentation) to purified biobutanol, according to embodiments of the inventions.

FIG. 2. A process flow diagram (PFD) for dewatering and contaminant removal from “biobutanol beer broth” obtained as a sparge from a fermentation vessel, according to embodiments of the inventions.

FIG. 3. Proton nuclear magnetic resonance (NMR) spectrum of dewatered bio-1-butanol obtained from an original “butanol beer broth” including 15-18 wt-% water. The original broth was obtained by fermentation of sugars using Clostridium bacteria, according to embodiments of the inventions.

It is to be understood that the foregoing general description and the following detailed description are exemplary and explanatory only and are not to be viewed as being restrictive of the invention, as claimed. Further advantages of this invention will be apparent after a review of the following detailed description of the disclosed embodiments, which are illustrated schematically in the accompanying drawings and in the appended claims.

### DETAILED DESCRIPTION OF EMBODIMENTS OF THE INVENTION

The invention generally relates to a process and fuel having biobutanol from fermentation processes is a direct precursor to 1-butene, thus, improvements in isolating biobutanol will decrease cost of the alternative JP-5 jet fuel.

1-butene can be utilized to make drop in JP-5 alternative fuels. Biobutanol from fermentation processes is a direct precursor to 1-butene, thus, improvements in isolating biobutanol will decrease cost of the alternative JP-5 jet fuel. Currently energy intense distillation methods are used to remove water from the biobutanol (as produced in fermentations). A rapid and quick drying with a brine solution can reduce the waste and cost of the associated with a lengthy distillation process and lead to increased yield of biobutanol.

Purification of butanol from fermentation processes has a long history, nearly as long at the initial discovery of the Acetone-Butanol-Ethanol process by Louis Pasteur in 1862. What has evolved as the most efficient purification process involves some kind of sparge using an inert carry stream. This can be carbon dioxide or simply water one practiced in the takes advantage of the butanol-water azeotrope. This azeotrope after collection then phase separates into an organic phase (top), the organic phase is often denoted as the “butanol beer broth,” which is decanted away from the bottom water layer, the latter is returned and can be used again as a sparging solvent.

Recently in patent application Ser. No. 12/018,216 (2008) the four general methods of purifying butanol were summarized as related to iso-butanol, although the methodology is applicable to both iso- and n-butanol.

The first method takes the organic layer including butanol and ˜5-20 water is simply further purified by fractional distillation. The first fractions are low boiling impurities followed by azeotropic (butanol-water) removal of the water and this is followed by collection of the purified butanol, typically 98% or greater. The butanol-water azeotrope is recycled and must be distilled again, utilizing process time and energy.

In method two, the butanol can be isolated from the fermentation medium using liquid-liquid extraction in combination with distillation. In this method, the butanol is extracted from the fermentation broth using liquid-liquid extraction with a suitable solvent. The butanol-containing organic phase is then distilled to separate the butanol from the solvent. This method can require large amounts of a flammable co-solvent and present difficulties with scale.

In method three, a distillation in combination with adsorption can be used to sequester butanol from the fermentation medium. For this process the fermentation broth containing the butanol is sparged near the azeotropic composition and then the remaining water is removed by use of an adsorbent, such as molecular sieves (Aden et al. Lignocellulosic Biomass to Ethanol Process Design and Economics Utilizing Co-Current Dilute Acid Prehydrolysis and Enzymatic Hydrolysis for Corn Stover, Report NREL/TP-510-32438, National Renewable Energy Laboratory, June 2002). This can require large amounts of absorbent since butanols typically carry 15-20 wt-% water once physically separated from the water phase.

A fourth approach is to combine a sparging process to first remove the butanol from the reactor and solids followed by a pervaporation process. As above, the fermentation broth containing the butanol (typically 1.0-2 wt-%) is sparged from the bioreactor at approximately azeotropic composition, after phase separation and the remaining water is removed by pervaporation, for example, through a hydrophilic membrane [Guo et al., J. Membr. Sci. 245, 199-210 (2004)]. Typically pervaporation processes are difficult for large scale operations and can be extremely costly on a commercial scale. When used before a sparging process and contact is made with the fermentation mixture, biofouling of the membrane is problematic.

Currently distillation alone is used to purify biobutanol on large scale production processes. This is done to remove ethanol, acetone, the predominance of water (˜15%), and other minor impurities. By using azeotropic distillation of the butanol followed by, physical separation, and then drying by exposure to a brine solution (saturated sodium chloride or other salts), one can have an almost continuous distillation process and eliminate the primary impurities of the biobutanol.

An aspect of the invention relates to a purification process of isolating biobutanol including;

removing biobutanol from a fermentation broth using at least one separation process and phase separating an organic layer and a concentrated biobutanol, dewatering the concentrated biobutanol and removing acid components with an aqueous saturated or near-saturated aqueous salt solution, chemically drying said biobutanol with at least one absorbent, and distilling the biobutanol to produce a purified biobutanol product.

Another aspect of the invention relates to a system that isolates biobutanol including; a fermentation vessel that removes biobutanol from a fermentation using a separation system that yields an organic layer of concentrated biobutanol, a dehydrating device that dewaters the concentrated biobutanol and removes acid components with an aqueous saturated or near-saturated aqueous salt solution, chemically drying device that the biobutanol with at least one absorbent, and distilling system that separates undesired components from the biobutanol to produce a purified biobutanol product.

Embodiments of the invention further include utilizing at least one inert gas during the separation process. In embodiments, the separation process is selected from the group consisting of a sparging process, membrane separation, and continuous feed by mechanical stirring of said broth. In embodiments, the dewatering includes passing said concentrated biobutanol over a stripper to remove acetone. In embodiments, the salt solution is a brine solution. In embodiments, the distilling system includes any fractionation process. In other embodiments, the distilling process includes any fractionation process having between 1 to 20 theoretical plates, or 10. In embodiments, the aqueous salt solution includes inorganic salts with sodium or potassium ions with negative ions of chloride, sulfate, carbonate, and bicarbonate at saturation. In other embodiments, salt solution includes inorganic salt solutions with sodium or potassium ions with negative ions of chloride, sulfate, carbonate, and bicarbonate. In other embodiments, the salt solution has a pH of about 8-10.

Embodiments of the invention include purified biobutanol product is about 97-99+% pure 1-butanol. In embodiments, the biobutanol is iso-butanol using collection method temperatures of about 107-110° C. In embodiments, the salt solution is 80-99% within saturation using sodium or potassium ions with negative ions of chloride, sulfate, carbonate, and biocarbonate. In other embodiments, the salt solution includes sodium or potassium ions with negative ions of chloride, sulfate, carbonate, and biocarbonate and is within 80 to 99% of saturation or near saturation, or 95%. In embodiments, the device that dewaters the biobutanol results in about 1-4% water remaining in the purified biobutanol product. In other embodiments, the dewatering of the biobutanol resulting in about 1-4% water remaining, or 2-3%, in the purified biobutanol product. Another aspect of the invention, a purified biobutanol product is obtained from the process defined above.

Embodiments of the invention further include utilizing at least one inert gas during the separation system. In embodiments, the separation system is selected from the group consisting of a sparging process, membrane separation, and continuous feed by mechanical stirring of the broth. In embodiments, the dehydrating device includes a stripper to remove acetone.

Embodiments of the invention will lead to an increase in production yield, simplify the equipment, lower energy use in the purification process, and concurrently eliminating acidic components. The brine solution-drying/purification method in this invention could be coupled with other techniques including sparging by carbon dioxide to remove and collect the biobutanol. The latter also requires a complicated distillation that could be mitigated by treatment with the brine solution.

Typical alternatives could be membrane technology, although this process can suffer from difficulty in running at large scale and continuous operation. To the best of my knowledge and having looked extensively at the open and patent literature, there does not appear to be any equivalent for this approach to purifying and isolating biobutanol. As it stands several distillations are required and involve reduced yields from losses in the distillation only processing. Embodiments of the invention would work for other alcohols like pentanol, which is also a target of workers in the fermentation business of making new biofuels.

An embodiment of the invention includes a method demonstrating a quick and efficient way to remove water and acids from butanol by contacting organic phase with a near saturated aqueous salt solution which rapidly dewaters the butanol and removes acid components. This can be followed by chemical drying using a minimum of absorbent and then distilled to afford pure butanol with exceptional mass yields and low energy consumption.

The butanol broth used in this invention can be prepared by one of several methods known to those skilled in the art of fermentation. Typically, once a maximum titer is achieved in the fermentation vessel the butanol is removed in some form of a sparging process whereby the butanol is concentrated and then phase separates as an organic layer that include by-products of fermentation process that may have organic chemicals/contaminants including, but not limited to, acetone, ethanol, furans, acetic acid, butyric acid, and various esters. Depending on the mixtures exact composition, 15-20 wt-% of water will also remain in the butanol. For this embodiment, 15-20 wt-% is the remaining water although content from 5 to 40 wt-% are acceptable.

The near-saturated or saturated salt solution used in embodiments of the invention is created by treating water with an excess amount of an inorganic salt. Typical inorganic salts that can be used in the invention include sodium or potassium ions with negative ions of chloride, sulfate, carbonate, and bicarbonate, but not limited to these combinations. A critical factor for the resulting salt solution is that it be sufficiently close to or at saturation. Once in process, the aqueous salt solutions can be brought to near saturation by evaporation water by an active heating/evaporation process, outdoor evaporation in open ponds, or in a recycling system using evaporation to remove water and concentrate the salt solution.

The salt solution is brought to a pH of 8-10, with 8 being another value in this embodiment.

The basic salt solution is brought in contact with the butanol sparge feed and mixed thoroughly by mechanical stirring for a period of 5 to 30 min, with 15 min in this embodiment. The top organic phase is the dewatered butanol with any acid contaminants removed and placed in the salt solution. It is passed over a stripper to remove acetone and then transferred through an optional secondary drying column and then on to a continuous distillation column for final fractionation. The bio-n-butanol when dried with exposure to an optional drying agent can bring the water content to below 1% (by weight). Collection of butanol from 113-117 deg C. affords 97-99+% pure 1-butanol with capture of 90-99% of the theoretical biobutanol included in the “biobutanol beer broth.” Similar results can be obtained for iso-butanol using an identical process except collecting 107-110 deg C. The bottoms in the final and only distillation are discarded in the process and typically represent 1-2 wt-% of the volume of purified butanol. They do have salt residue that can be recycled.

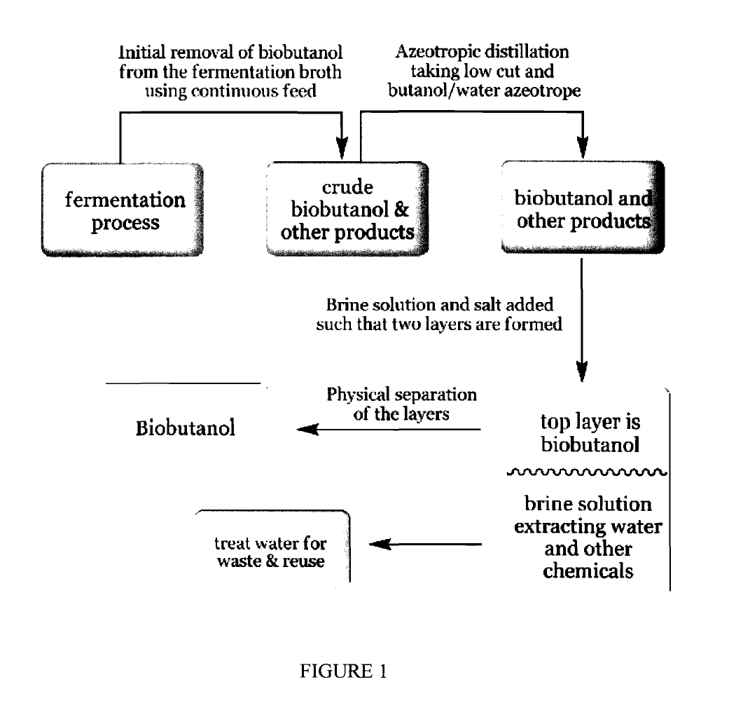

FIG. 1. A general overview of process going from sugars (fermentation) to purified biobutanol.

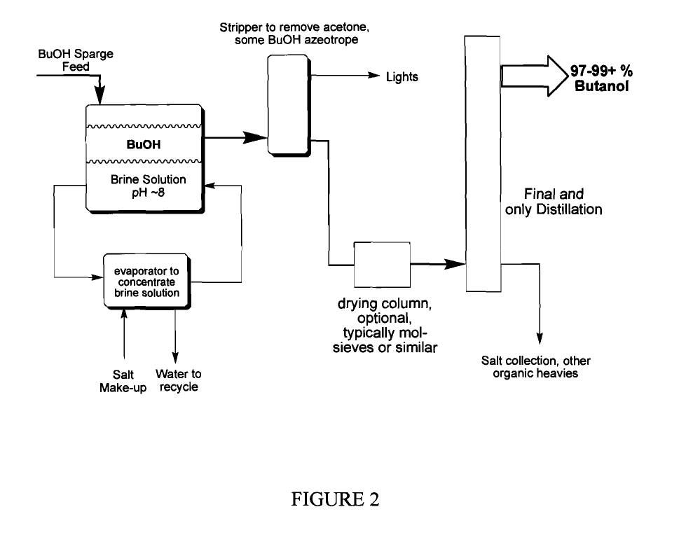

FIG. 2. A process flow diagram (PFD) for dewatering and contaminant removal from “biobutanol beer broth” obtained as a sparge from a fermentation vessel.

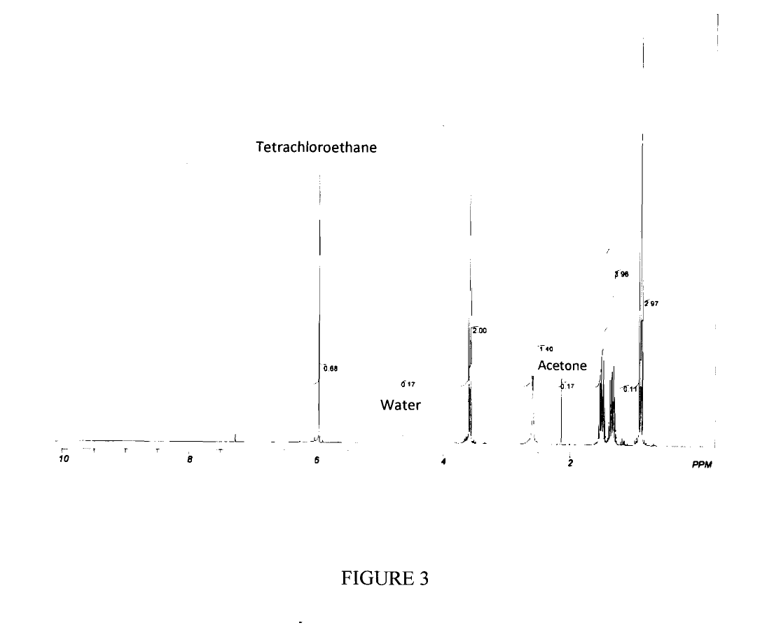

FIG. 3. Proton nuclear magnetic resonance (NMR) spectrum of dewatered bio-1-butanol obtained from an original “butanol beer broth” including 15-18 wt-% water. The original broth was obtained by fermentation of sugars using Clostridium bacteria.

### EXAMPLE

1) Bio-n-butanol is removed from the fermentation vessel by a process of a) sparging with an inert gas, b) membrane separation, c) or some other physical means that will remove the bacteria, yeast, or fungi and remove water and concentrate the biobutanol component.

2) The bio-n-butanol azeotrope (˜55% butanol, 45% water, b.p. 97° C.) is carefully distilled using an apparatus with sufficient theoretical separation plates to minimize additional water (bp 100 deg C.) co-distilling with the azeotrope. In addition to the bio-n-butanol azeotrope other organics and azeotropes will lead the distillation run. They can be discarded or left in the distillate and will be removed in steps 3 and 4.

### Version A

3) After settling, the azeotrope will form two layers, sodium chloride is added at a sufficient amount to saturate the water layer, the layers are separated and the butanol is isolated by a quick physical separation and can be used in certain processes, for example, dehydration to form bio-1-butene.

### Version B

3) The water layer can be separated (discarded or used in a slower secondary process to remove the butanol) from the biobutanol, leaving a water saturated biobutanol. This biobutanol will be treated with a saturated salt solution, typically in the range of 10-50% of the volume of the butanol volume, thus removing most of the water. The layers are separated by quick physical separation and the brine solution can be strengthened (i.e. salt added and/or water evaporated off) and used many times. An optional secondary process to remove additional water can be utilized. This can be 3A mol-sieves or a related solid-phase drying agent that can be recycled in service by heating or similar. See FIG. 3 for a typical dried bio-n-butanol sample using this process.

4) Depending on the biobutanol specs, a final distillation to remove last traces of water, ethanol, acetone, and other minor impurities may be necessary. For many applications, this biobutanol may be of sufficient purity, for example use in preparing biobutene that is useful in making diesel and jet fuels and biolubricants.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

While the invention has been described, disclosed, illustrated and shown in various terms of certain embodiments or modifications which it has presumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

What is claimed is:

1. A purification process of isolating biobutanol, comprising:
removing biobutanol from a fermentation broth using at least one separation process and phase separating an organic layer and a concentrated biobutanol;
dewatering said concentrated biobutanol and removing acid components with an aqueous saturated or near-saturated aqueous salt solution;
chemically drying said biobutanol with at least one absorbent; and
distilling said biobutanol to produce a purified biobutanol product.

2. The process according to claim 1, further comprising utilizing at least one inert gas during the separation process.

3. The process according to claim 1, wherein said separation process is selected from the group consisting of a sparging process, membrane separation, and continuous feed by mechanical stirring of said broth.

4. The process according to claim 1, wherein said dewatering includes passing said concentrated biobutanol over a stripper to remove acetone.

5. The process according to claim 1, wherein said salt solution is a brine solution.

6. The process according to claim 1, wherein said distilling process includes any fractionation process having between 1 to 20 theoretical plates, or 10.

7. The process according to claim 1, wherein said aqueous salt solution includes inorganic salts with sodium or potassium ions with negative ions of chloride, sulfate, carbonate, and bicarbonate at saturation.

8. The process according to claim 1, wherein said salt solution having a pH of about 8-10.

9. The process according to claim 1, wherein said purified biobutanol product is about 97-99+% pure 1-butanol.

10. The process according to claim 1, wherein said biobutanol is iso-butanol using collection method temperatures of about 107-110° C.

11. The process according to claim 1, wherein said salt solution includes sodium or potassium ions with negative ions of chloride, sulfate, carbonate, and biocarbonate and is within 80 to 99% of saturation or near saturation, or 95%.

12. The process according to claim 1, wherein said dewatering of said biobutanol resulting in about 1-4% water remaining, or 2-3%, in said purified biobutanol product.

13. A purified biobutanol product obtained from the process defined in claim 1.

14. A system that isolates biobutanol, comprising:
a fermentation vessel that removes biobutanol from a fermentation using a separation system that yields an organic layer of concentrated biobutanol;
a dehydrating device that dewaters said concentrated biobutanol and removes acid components with an aqueous saturated or near-saturated aqueous salt solution;
chemically drying device that said biobutanol with at least one absorbent; and
distilling system that separates undesired components from said biobutanol to produce a purified biobutanol product.

15. The system according to claim 14, further comprising utilizing at least one inert gas during the separation system.

16. The system according to claim 14, wherein said separation system is selected from the group consisting of a sparging process, membrane separation, and continuous feed by mechanical stirring of said broth.

17. The system according to claim 14, wherein said dehydrating device includes a stripper to remove acetone.

18. The system according to claim 14, wherein said salt solution is a brine solution.

19. The system according to claim 14, wherein said distilling system includes any fractionation process.

20. The system according to claim 14, wherein said salt solution includes inorganic salt solutions with sodium or potassium ions with negative ions of chloride, sulfate, carbonate, and bicarbonate.

21. The system according to claim 14, wherein said salt solution having a pH of about 8-10.

22. The system according to claim 14, wherein said purified biobutanol product is about 97-99+% pure bio-1-butanol.

23. The system according to claim 14, wherein said biobutanol is iso-butanol using collection method temperatures of about 107-110° C.

24. The system according to claim 14, wherein said salt solution is 80-99% within saturation using sodium or potassium ions with negative ions of chloride, sulfate, carbonate, and biocarbonate.

25. The system according to claim 14, wherein said device that dewaters said biobutanol results in about 1-4% water remaining in said purified biobutanol product.
